(19) United States
(12) Patent Application Publication
(10) Pub. No.: US 2013/0109098 A1
(43) Pub. Date: May 2, 2013

(54) BIOSECURE GENETICALLY MODIFIED ALGAE

(71) Applicants: F.C. Thomas Allnutt, Glenwood, MD (US); Bradley Lynn Postier, St. Charles, MO (US); Richard T. Sayre, Webster Groves, MO (US); Daniel A. Coury, University Heights, OH (US); Anil Kumar, St. Louis, MO (US); Andrew Swanson, Cleveland Heights, OH (US); Mark Scott Abad, Webster Groves, MO (US); Zoee Gokhale Perrine, St. Louis, MO (US)

(72) Inventors: F.C. Thomas Allnutt, Glenwood, MD (US); Bradley Lynn Postier, St. Charles, MO (US); Richard T. Sayre, Webster Groves, MO (US); Daniel A. Coury, University Heights, OH (US); Anil Kumar, St. Louis, MO (US); Andrew Swanson, Cleveland Heights, OH (US); Mark Scott Abad, Webster Groves, MO (US); Zoee Gokhale Perrine, St. Louis, MO (US)

(73) Assignee: PHYCAL, INC, Highland Heights, OH (US)

(21) Appl. No.: 13/808,285

(22) PCT Filed: Jul. 6, 2011
(86) PCT No.: PCT/US2011/043009

§ 371 Date: Jan. 4, 2013

Related U.S. Application Data
(60) Provisional application No. 61/361,668, filed on Jul. 6, 2010.

(51) Int. Cl. C12N 15/79

(52) U.S. Cl. CPC C12N 15/79
USPC 435/471; 435/257.2

(57) ABSTRACT

Biosecure algae and methods for preparing biosecure algae that have a substantially decreased capability to survive in a natural environment are described. The methods include transforming a genetically modified alga to include an essential gene that is operably linked to a promoter system that is active only in the presence of an inducer compound, transforming the genetically modified alga to include a lethal gene that is operably linked with a promoter system that is inactive only in the presence of a repressor compound. The biosecure algae are only able to survive in an artificial algae culture that includes factors or conditions not found in a natural environment.

### CROSS-REFERENCE TO RELATED APPLICATION

This application claims priority to, and any other benefit of, U.S. Provisional Patent Application Ser. No. 61/361,668, entitled BIOSECURE PRODUCTION FROM ALGAE and filed Jul. 6, 2010, the entire disclosure of which is fully incorporated herein by reference.

### BACKGROUND

There is little debate about the need for an affordable, renewable feedstock to replace geologically occurring crude oil as a source for transportation fuels. Renewable fuels would significantly increase global energy security, and could also provide significant environmental benefits. However, current renewable fuels (e.g., biodiesel from edible oils and ethanol) are not affordable in the sense that they require significant operating subsidies. They also compete directly with the food supply causing food price inflation. In addition, the large scale farming of crop-based biofuels contributes to soil erosion and agricultural runoff.

A combination of instability in oil-producing regions of the world, rapid economic growth in the developing world, strong demand in the developed world, and finite fossil-fuel supplies recently pushed energy prices to new heights. The political instability in the Middle East and other oil producing nations, such as Russia and Venezuela, has made western nations keenly aware of the need for secure sources of feedstock for the production of transportation fuels. Finally, the attention given to the role of greenhouse gases in climate change has increased the importance of developing a renewable biofuel feedstock that does not directly compete with the food supply. The current global recession has moderated petroleum prices, which increases the importance of using a low cost feedstock to produce fuel at a competitive price. Renewable fuels derived from algae (algal oil and algal biomass derived fuels) are increasingly considered the best option to address all these concerns simultaneously.

New biodiesel processes will accept any plant oil, including algal oil, for transesterification into biodiesel that meets the latest ASTM specifications. Additionally, both UOP (the world's leading refining technology company) and GE Research have developed processes to refine any plant oil, including algal oil, into drop-in replacements for gasoline, jet fuel, diesel, and ethylene (the feedstock for many petrochemicals). The technology to refine plant lipids such as algal oil into transportation fuels is thus available, but is not currently used commercially because the plant and algal feedstocks cost much more than petroleum. An industrial scale supply of algal oil will find a ready market if it can be provided at a competitive cost. There is growing recognition that microalgae may be one of the most efficient photosynthetic organisms for the production of lipids and oil-based biofuels (Chisti, Biotech Adv 25: p. 294-306 (2007)). The key to wide-spread adoption of algal oil feedstock is cost competitiveness. One of the methods available for providing a more cost-effective algal feedstock for biofuel production is to genetically modify the algae to produce higher levels of oil.

Algal biomass derived fuels also benefit from the above data. Methods to convert algal biomass into syn gas and alternative fuels have been tested at laboratory scale and are benefitting from increased emphasis in both academic and industrial research and development.

All photosynthetically derived algal biofuels provide an advantage versus fossil fuels in that they capture carbon dioxide from the air or from industrial emissions and recycle the carbon into additional fuels and other bioproducts (e.g., bioplastics and chemicals). Such a cycle, while not perfect in capturing all emitted carbon, increases the efficiency of the energy production process and lowers the overall carbon footprint of energy consumed. The same can be said of bioderived plastics and chemicals.

Production of algal biofuels is currently limited by the genetic potential of the strains being used to grow quickly and/or to produce lipid. Genetic engineering could introduce new capabilities to produce more economical biofuels, but the use of genetically modified organisms (GMOs) requires that biosecurity or containment be assured to prevent their escape into the environment with the attendant possible unintended damage. Mechanical containment in enclosed photobioreactors is not sufficient because of the need to remove and process large quantities of biomass to produce biofuels. Additionally, the commodity pricing of the products produced may make photobioreactor production cost prohibitive. Open pond systems offer enticing economic potential but providing algal biosecurity using mechanical means is not possible for these types of systems. Great strides have been made in algal biomass production systems (mass culture) over the past thirty years. Concomitantly, advances in the ability to genetically manipulate algae promise to allow the achievement of higher lipid and biomass production yields. Optimization of algal biofuel economics using only mechanical and chemical engineering improvements will become incrementally harder as the continuous improvement process continues. Significant cost reductions will eventually require genetic manipulation of the production strains using molecular biological techniques. The need therefore exists to provide biosecure production of genetically modified algae, particularly in the context of open pond systems.

### SUMMARY OF THE INVENTION

The present invention provides biosecure alga, and methods of making and culturing biosecure alga. Embodiments of the present invention also address the need for the economic production of algal biofuels and bioproducts. The underlying technical need addressed is the secure use of algae or other organisms that are genetically modified for increased lipid and biomass production (or other economically valuable trait). Adding biosecurity to genetically modified algae that provide increased lipid and biomass growth in open photobioreactors provides two major cost improvements: 1) the increased production of lipid and biomass per hectare and 2) the secure use of the lowest cost open pond mass culturing systems.

Currently large-scale production of genetically modified organism (GMO) algae is not done in open pond systems or, in fact anywhere. The real advantage to algae is their photosynthetic growth form (requiring sunlight and no fixed carbon), yet this advantage works against their use in large-scale GMO production. This is so because if the GMO algae escaped and were carried to a suitable environment they could readily grow in unwanted areas. A biosecurity system rendering GMO algae incapable of survival out of the production ponds or photobioreactors allows use of the large body of information on pond or photobioreactor culture to make commercial open pond or photobioreactor culture of GMO algae practical and environmentally responsible. Such a biosecurity system is amenable to a large open outdoor system as well as enclosed photobioreactors. In both cases, the biosecurity or biocontainment provided in the modified cell would prevent escaped algal cells from multiplying once they are in an environment that does not contain the chemical inducers necessary to turn on or turn off the engineered promoters or other physical conditions required for survival of the GMO algae as described herein.

### BRIEF DESCRIPTION OF THE FIGURES

The present invention may be more readily understood by reference to the following drawing wherein:

FIG. 1 provides a schematic representation of a biosecure alga that has been modified to include the barnase/barstar toxin/antitoxin pair and how it interacts with an inducing compound.

To illustrate the invention, several embodiments of the invention will now be described in more detail. Skilled artisans will recognize the embodiments provided herein have many useful alternatives that fall within the scope of the invention.

### DETAILED DESCRIPTION OF THE INVENTION

The present invention provides systems and methods for improved biosecurity for algal production or culturing systems that utilize genetically modified organisms. This is particularly applicable to methods for commodity products such as biofuel produced in open or semi-open production systems such as, but not limited to, those using microalgae in open ponds, raceways, or photobioreactors.

### DEFINITIONS

Unless otherwise defined, all technical and scientific terms used herein have the same meaning as commonly understood by one of ordinary skill in the art to which this invention pertains. In case of conflict, the present specification will control.

The terminology as set forth herein is for description of the embodiments only and should not be construed as limiting of the invention as a whole. Unless otherwise specified, “a,” “an,” “the,” and “at least one” are used interchangeably. Furthermore, as used in the description of the invention and the appended claims, the singular forms “a”, “an”, and “the” are inclusive of their plural forms, unless contraindicated by the context surrounding such.

The terms “comprising” and variations thereof do not have a limiting meaning where these terms appear in the description and claims.

Also herein, the recitations of numerical ranges by endpoints include all numbers subsumed within that range (e.g., 1 to 5 includes 1, 1.5, 2, 2.75, 3, 3.80, 4, 5, etc.).

The phrase “genetically modified algae,” as used herein, refers to algae whose genetic material has been altered using genetic engineering techniques so that it is no longer a “wild “type” organism. An example of a genetically modified alga is a transgenic alga that possess one or more genes that have been transferred to the algae from a different species. Accordingly, as used herein, transgenic algae are genetically modified algae.

The term “transformation” is used to refer to the uptake of foreign DNA by cell such as an algae cell. A cell has been “transformed” when exogenous DNA has been introduced inside the cell membrane. The term refers to both stable and transient uptake of the genetic material.

The term “expression vector” refers to any type of genetic construct comprising a nucleic acid coding for a RNA capable of being transcribed. Examples of expression vectors include plasmids, viruses, cosmids, and artificial chromosomes. Expression vectors all include an origin of replication, a multicloning site, and a selectable marker. Expression vectors include an expression cassette including a variety of control sequences, structural genes (e.g., genes of interest), and nucleic acid sequences that serve other functions as well.

The term “Heteroboost™” and “heterotrophic boost” refer to changing the trophic state of phototrophic or mixotrophically grown algae to photoheterotrophic or purely heterotrophic growth. As used herein “photoheterotrophic growth” is growth where light is added in a very small amount such that its action is not to induce photosynthesis but to light poise, activate, or otherwise influence the metabolic activity of the algae. As used herein “mixotrophic growth” refers to growth of algae in the light in the presence of at least a small amount of fixed carbon such that photosynthesis and heterotrophic activities occur simultaneously or sequentially in the same reactor or growth system.

With regard to the alga species recited herein, it is noted that the taxonomy of algal species is in constant flux. Therefore it is possible that genera, species, and strains will change their names as time progresses. Where possible, alternative strain names are provided. However, it is anticipated that the current status of genus and species designations will change over time and the invention will maintain its relevance to the strains whatever their eventual designation. A current example is the renaming of Chlorella protothecoides as Auxenochlorella protothecoides. For the purposes of this invention they should be treated as the same organism.

The term “algae” is commonly used as the plural of the term “alga”. The term “algal” is usually used as an adjective. However, it is common practice to use these terms interchangeably. Herein, the terms are not used to limit the specific application to a particular number or application and should be interpreted to include the broadest coverage of the claim.

The present invention provides methods of malting a biosecure alga. The biosecure alga is made by modifying an existing alga using recombinant DNA techniques to incorporate one or more genes into the alga which reduce its ability to survive in a natural environment. A biosecure alga, as defined herein, is an alga whose ability to survive is a natural environment is substantially reduced in comparison to a non-biosecure alga. The alga is modified to be biosecure in order to substantially decrease the likelihood that a genetically modified alga will be able to survive and reproduce outside of the confines of the artificial environment in which it is intended to be grown in a production process. Preferably a biosecure alga is one whose ability to grow in the natural environment is reduced by at least 90%. More preferably, the biosecure alga is unable to grow at all in a natural environment, and even more preferably the biosecure alga will die when placed in a natural environment.

In order to culture an alga in a biosecure manner, the alga is genetically modified to decrease its ability to survive should it ever be released from the algae culture system into the surrounding “natural environment.” The natural environment, as defined herein, includes locations where the algae may grow that are not man-made, such as naturally occurring ponds. Viewed another way, the biosecure alga is modified so that it can only survive in an intended artificial environment, such as a biofuel or bioproduct production facility, where one or more factors are provided that are absent in the natural environment that allow the biosecure alga to survive in the artificial environment.

To prevent transgenic algae from moving into the natural environment, “sentinel” type technologies that will preclude the growth and reproduction of algae that escape the intended culture system can be used. Most embodiments of the biosecurity system described herein are based on conditional gene expression systems that will control the expression of an essential gene or genes based on the presence of an inducer chemical compound that is not typically found in substantial quantities in the natural environment for algae. Alternatively, the sentinel system inhibits expression of a lethal gene unless the organism is removed from the controlled culture system of the production facility whereupon the lethal gene is expressed and kills or inhibits the growth the escaped algal cell. The invention also contemplates several additional variants that deviate from these basic approaches, including stacking of multiple, redundant biosecurity strategies, for making a biosecure alga.

In addition to direct control of an essential or lethal gene, several more indirect approaches can be taken. For example, the alga can be transformed to include a toxin gene under the control of a constitutive promoter along with a an antitoxin linked to a promoter that is active only in the presence of an inducer compound. The antitoxin inhibits the activity of the toxin so long as it is expressed. Alternatively, the promoter may be used to control expression of a gene encoding a regulatory protein that has an effect on the activity or expression of an essential or lethal gene. In a further embodiment, the promoter may be linked to a gene whose expression affects transport within the alga cell. For example, the promoter may be linked to a holin gene whose expression provides holes in the cell membrane that allow constitutively expressed lytic enzymes to pass through the cell membrane and reach the cell wall, thereby eliciting cell lysis.

In other embodiments, the algae are modified to require the presence of other types of factors in the artificial environment. For example, the algae can be modified to have increased vulnerability to ultraviolet radiation, requiring that they be grown in an environment that is wholly or partially shielded from ultraviolet radiation. Alternately, the algae can be modified to constitutively express a lethal gene whose activity is inhibited by microRNA and/or siRNA directed to that lethal gene or its promoter that are included in the alga culture medium in the artificial environment.

A wide variety of algae are suitable for genetic modification. Among these are dinoflagellates (e.g., Ampidinium, Symbidinium), diatoms (e.g., Phaeodactylum, Cyclotella, Navicula, Cylindrotheca, Thalassiosira), green algae (e.g. Chlamydomonas, Chlorella, Haematococcus, Dunaliella), red algae (e.g., Kappaphycus), macroalgae (e.g., Ulva), and bluegreen algae (e.g., Synechocystis, Synechococcus, Anabaena, Nostoc). Methods and protocols for the genetic modification of algae have been described in the literature and are incorporated herein by reference (Leon & Fernandez Advances in Experimental Medicine and Biology, Ch. 1, 616 1-129 (2007)); Packer & Glazer, Meth Enzymol 167, p. 1-910 (1988); Bryant, The molecular biology of Cyanobacteria. Advances in Photosynthesis, Kluwer Academic Publishers. 880 pp (1994)).

Algae that are genetically modified can be selected, for example, from the group consisting of cyanophyta, rhodophyta, heterokontophyta, haptophyta, cryptophyta, dinophyta, euglenophyta, and chlorophyta. The genetically modified alga can also be a species selected from the group consisting of Auxenochlorella sp., Parachlorella sp., Chlamydomonas sp., Chlorella sp., Nannochloropsis sp., Synechocystis sp., Synechococcus, Anabaena sp., Cyclotella, Phaeodactylum sp., Crypthicodineum sp., Schizochytridum sp., Haematococcus sp., Arthrospira(Spirulina) sp. and Dunaliella sp.

The need for biosecurity typically arises as a result of the use of genetically modified organisms. The genetically modified algae of the present invention are typically modified in a manner that increases their usefulness for biofuel production. For example, the alga may be genetically modified to provide increased production of lipid, which is a biofuel precursor. However, biofuel production can also be improved by genetically modifying the alga to have increased resistance to predators, increased ability to survive oil extraction, increased growth rates, improved capture of carbon dioxide, increased ability to capture light for use in biosynthesis, or a wide variety of other modifications that can increase biofuel production. See for example WO 2009/073822, which describes molecular approaches for the optimization of biofuel production and is incorporated herein by reference. Alternately, or in addition, the genetically modified alga of the present invention may be engineered to have increased tolerance or resistance to abiotic stresses from outdoor environmental conditions or to biotic stresses arising from competitive or predatory contaminating organisms coinhabiting the pond environment. Additionally, the alga can be modified to increase the production of high value co-products (e.g., pigments and secondary metabolites) that can reduce the overall cost of the process by providing additional revenue.

For embodiments directed to biofuel production, the algae of the present invention are preferably oleaginous algae. An oleaginous alga is an algal species that can, under known conditions, accumulate a significant portion of its biomass as lipid. For example, embodiments of oleaginous algae are algal species that are capable of accumulating at least 10%, at least 20%, at least 30%, at least 40%, or at least 50% of their biomass as lipid. Suitable oleaginous algae species can be found in the Bacillariophyceae, Chlorophyceae, Cyanophyceae, Xanthophyceaei, Chrysophyceae, Chlorella, Crypthecodinium, Schizocytrium, Nannochloropsis, Ulkenia, Dunaliella, Cyclotella, Navicula, Nitzschia, Cyclotella, Pareitochloris, Phaeodactylum, and Thaustochytrid classes and genera. A preferred genus of oleaginous algae is Chlorella, which includes numerous species capable of accumulating about 55% of their total biomass as lipids. See for example Miao & Wu, Journal of Biotechnology, 110, p. 85-93 (2004). Suitable Chlorella species include Chlorella vulgaris, Chlorella (Auxenochlorella) protothecoides, Chlorella sorokiniana, and Chlorella kessleri.

The algae species used form a part of an algae culture. An algal culture, as used herein, refers to one or more algal species living in an environment that generally enables their survival and growth. The algae culture may either an artificial algae culture, such as that found in a biofuel production facility, or it can be a natural algae culture found in the algae's natural environment or a non-native environment. Note that a natural algae culture will not support the growth of a biosecure algae, as described herein. The culture conditions required for various algae species are known to those skilled in the art. Examples of the components of an algal culture include water, carbon dioxide, minerals and light. However, the components of an algal culture can vary depending on the algae species, and whether or not conditions for autotrophic, mixotrophic, Photoheterotrophic, or heterotrophic growth are desired. For autotrophic growth, the algae culture will require CO₂ and light energy (e.g., sunlight), whereas heterotrophic growth requires organic substrates such as sugar for the growth of the algae culture, and can be carried out in the absence of light energy. Autotrophic, mixotrophic, photoheterotrophic, and heterotrophic algae cultures are all within the scope of the present invention.

An algae culture requires that appropriate temperature conditions be maintained, and preferably that the culture is mixed to provide even access to nutrients and/or light. While algae can grow in non-aqueous environments, algal culture as referred to herein is an algal culture in an aqueous environment, and therefore includes algae growing a liquid or submerged in liquid. Preferably the algal culture is a monoculture including a single algae species, or at least is intended as such, taking into account possible contaminating predators and competitors. Use of a monoculture makes it easier to provide optimal culture conditions, and can simplify growing and processing the algae in other ways. Alternatively, a consortium of algal species or strains could be used as appropriate. Open pond systems and/or systems where a biofilm is maintained on the surface often include a consortia of organisms that are maintained over time for production of biofuels and bioproducts. Open pond systems including a plurality of organisms, as well as other algal culture systems that include a plurality of algal species, are also within the scope of the present invention.

Methods for the transformation of various types of algae are known to those skilled in the art. See for example Radakovits et al., Eukaryotic Cell, 9, 486-501 (2010), which is incorporated herein by reference. The transformation of the chloroplast genome was the earliest method and is well documented in the literature (Kindle et al., Proc Natl Acad. Sci., 88, p. 1721-1725 (1991)). A variety of methods have been used to transfer DNA into microalgal cells, including but not limited to agitation in the presence of glass beads or silicon carbide whiskers, electroporation, biolistic microparticle bombardment, and Agrobacterium tumefaciens-mediated gene transfer. A preferred method of transformation for the present invention is biolistic microparticle bombardment, carried out with a device referred to as a “gene gun.”

Different regions of the alga may be targeted for transformation in different embodiments of the invention. Target regions include the nuclear genome, the mitochondrial genome, and the chloroplast genome. The preferred target region can vary depending on the gene being expressed. For example, if an alga has been modified to express a lethal gene that is obtained from a bacterium, it may be preferable to express the lethal gene in the chloroplast or mitochondrion, as these organelles evolved from bacteria and retain many similarities. This can be achieved using a chloroplast expression vector that employs 2 intergenic regions of the chloroplast genome that flank and drive the site-specific integration of a transgene cassette (5′ untranslated region, or 5′UTR followed by the coding sequence of the protein to be expressed which can drive the biological function desired, followed by a 3′ UTR). The 5′UTR contains a cis acting site that allows docking of the RNA Polymerase that drives transcription of the transgene. The 3′UTR contains sequence that allows for the correct termination of the transcription by RNA Polymerase. However, in other cases, such as when the essential or lethal gene has an effect in various regions of the cell, it may be preferable to express the gene in the nucleus if the algae is eukaryotic. This can be achieved with a gene cassette that employs a eukaryotic promoter sequence upstream of the protein coding sequence and a eukaryotic termination sequence downstream of the protein coding sequence.

Genetically modified algae can be transformed to include an expression cassette. An expression cassette is made up of one or more genes and the sequences controlling their expression. The three main components of an expression cassette are a promoter sequence, an open reading frame expressing the gene, and a 3′ untranslated region that usually contains a polyadenylation site. The cassette is part of vector DNA used for transformation. The promoter is operably linked to the gene expressed represented by the open reading frame.

The term “operably linked” refers to the arrangement of various polynucleotide elements relative to each other such that the elements are functionally connected and are able to interact with each other. Such elements may include, without limitation, a promoter, an enhancer, a polyadenylation sequence, one or more introns and/or exons, terminator, and a coding sequence of a gene of interest to be expressed. The nucleic acid sequence elements, when operably linked, act together to modulate the activity of one another, and will affect the level of expression of the gene of interest (e.g., an essential gene or a lethal gene). Modulate means increasing, decreasing, or maintaining the level of activity of a particular element. The position of each element relative to other elements may be expressed in terms of the 5′ terminus and the 3′ terminus of each element.

The term “promoter” refers to a nucleic acid sequence that regulates, either directly or indirectly, the transcription of a corresponding nucleic acid coding sequence to which it is operably linked. The promoter may function alone to regulate transcription, or, in some cases, may act in concert with one or more other regulatory sequences such as an enhancer, silencer, terminator, or transcription factor to regulate transcription of the transgene. When the promoter is acting in conjunction with other factors such as a transcription factor, it is referred to herein as a promoter system. The promoter comprises a DNA regulatory sequence, wherein the regulatory sequence is derived from a gene, which is capable of binding RNA polymerase and initiating transcription of a downstream (3′-direction) coding sequence.

A promoter generally comprises a sequence that functions to position the start site for RNA synthesis. The best-known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. The spacing between promoter elements frequently is flexible, so that promoter function is preserved when elements are inverted or moved relative to one another To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

A promoter may be one naturally associated with a nucleic acid sequence, as may be obtained by isolating the 5′ non-coding sequences located upstream of the coding segment and/or exon. Such a promoter can be referred to as “endogenous.” Alternatively, certain advantages may be gained by positioning the coding nucleic acid segment under the control of a recombinant or heterologous promoter, which refers to a promoter that is not normally associated with a nucleic acid sequence in its natural environment. Such promoters may include promoters of other genes, and promoters isolated from any other virus, or prokaryotic or eukaryotic cell, and promoters not “naturally occurring,” i.e., containing different elements of different transcriptional regulatory regions, and/or mutations that alter expression. Control sequences that direct transcription and/or expression of sequences within non-nuclear organelles such as mitochondria, chloroplasts, and the like, can be employed as well.

A transcription factor is a protein that binds to specific DNA sequences and thereby controls the transfer (or transcription) of genetic information from DNA to mRNA. Transcription factors perform this function alone or with other proteins in a complex, by promoting (as an activator), or blocking (as a repressor) the recruitment of RNA polymerase, which is the enzyme that performs the transcription of genetic information from DNA to RNA, to specific genes. A defining feature of transcription factors is that they contain one or more DNA-binding domains that attach to specific sequences of DNA adjacent to the genes that they regulate.

Numerous embodiments of the invention provide biosecure algae that include a promoter that is active only in the presence of an inducer compound. Typically, the promoter system includes a transcription factor that binds to the inducer compound, which then binds to a consensus binding sequence near the promoter, resulting in activation of the promoter and expression of the associated gene. Examples of suitable transcription factors include norR, DNR, NmIR, and cbbR.

Inducer compounds are preferably low cost compounds that can be economically used to control expression of a gene or number of genes, at least one of which is required for growth of the alga (e.g., an essential gene) in a genetically modified alga to ensure that the alga can only grow in the presence of the inducer. The inducer is incorporated into the algal culture system to allow growth of the alga. If the alga is removed or escapes from the production system, the inducer will no longer be present and therefore the algae will therefore not express the required gene and will either die or cease growing. The overall effect of this system will be to ensure that the genetically modified organisms cannot escape and damage the environment. To be an effective system the inducer compound must not be present in the normal growth environment in a biologically significant quantity. Preferably, the inducer is already present or can be added in a very small quantity to the production system.

Inducer compounds for controlling algal protein expression are known, but these known inducers are typically expensive compounds (e.g., isopropyl β-D-1-thiogalactopyranoside, β-glucuronidase, or antibiotics) designed for research purposes. The inducers envisioned by this invention are preferably low cost compounds that are already present or can be added to the huge volumes of algal culture medium at low concentration.

For example, soybean processing produces a number of waste flavonoids that can function as inducer compounds. These inexpensive chemicals can be added into algae ponds to induce expression of genes that are required for growth in the GMO alga (i.e., essential genes). Such a biosecurity approach has been utilized for bacterial and yeast genetic systems by making auxotrophic mutants that required specific carbon sources, amino acids, or nucleotides for growth (Giga-Hama et al. Appl Biochem. 46 (pt 3):147-55 (2007); Li et al., Lett. Appl. Micro 37(6):458-62 (2003); Shen et al. Plant Cell. 5, p. 1853-63 (1993)). Such an approach is also contemplated for use in the instant invention.

Inexpensive chemicals with specific receptors on the algal cell membrane or nucleic acid can function as inducer compounds. One example would be a metal chelating induction system. One could use a non-toxic metal as an inducer (e.g., of zinc ion, copper ion, cadmium ion, or iron ion) for this approach (Cousins Annu Rev Nutr 14, p. 449-469 (1994)). Other examples of inducer/promoter systems are the phytochellatin gene (Abner et al., Limnol Oceanogr 40, p. 658-669 (1995)) and the promoter for the copA gene of Escherichia coli (Rensing et al., PNAS 97, p. 652-656 (1999)). Unique and non-digestible sugars such as inulin and chitin might also be useful as inducers as promoters induced by these compounds are also known.

As noted above, metal ions can be used as inducer compounds to control the growth of genetically modified algae. The yeast ace1 gene encodes a metallo-regulatory transcription factor ACE1 that has been used to place genes under the control of copper ions in plant cell systems (Mett et al., Transgenic Res., 5, p. 105-113 (1996); Makenzie et al., Plant Physiol 116, p. 969-977 (1998)). Placing this transcription factor in front of a promoter (whether constitutive or not) can be used as another method to control expression of a gene or genes that are necessary for algal survival in their culture system. The approach would parallel that described in Example 1 later herein, except that in front of the psbA gene's normal promoter a transcription control element would be used to prevent transcription without the presence of copper ion at the required level. Cadmium and iron-stress-inducible gene expression are also possible (Sayre R T, Planta, 215, p. 1-13 (2002))

Low cost compounds such as phenolics, flavonoids, lectins, binding proteins, and organic molecules that are residuals found in agricultural processing wastes and can be utilized as inducers for specific promoters. Such agricultural waste streams can be obtained from dairy processing (e.g., whey components), fermentation byproducts (e.g., yeast), corn processing (e.g., corn steep liqueur), soybean processing, and various other processes.

Another group of compounds that can be useful as inducer compounds are those that naturally leach from the sides of a typical artificial algae pond with a plastic liner. These algae pond liner (e.g., plastic) materials are not typically present in the natural environment for the algae. These inducer compounds include phthalates, polyvinyl chloride, polyethylene resins, and fiberglass. Burkholderia cepacia is capable of metabolizing phthalate. One gene ophE is specifically induced by phthalate. (Zylstra, Journal of Bacteriology, p. 3069-3075, (1999))

Another source of inducer compounds include those that are present in flue gas from an industrial facility such as a power plant. Flue gas often includes high levels of carbon dioxide, which can be used to stimulate algae growth. The flue gas also includes other compounds such as nitric oxide or carbon monoxide that can function as inducer compounds.

Another source of inducer compounds includes environmentally safe and approved pesticides such as herbicides, insecticides, and the like, which may be provided in an artificial algae culture. Preferably these are used in very small quantities to maintain the induction of an essential gene in the artificial algae culture. If the algae escape from the artificial algae culture, lack of expression of the essential gene (or induction of a lethal gene) results in cytotoxicity or cytostasis of the genetically modified alga.

In another embodiment, solvent-derived inducing compounds are used. For biofuels production the process of producing lipid during photosynthetic growth is improved by “milking” the algae. That is continuously extracting lipid from a fraction of the pond using hexane, decane, isoparaffins, or other organic solvents. One drawback to this process is the constant exposure of the algae to low levels of solvent. While many strains of algae have demonstrated the ability to tolerate hexane and other alkane solvents it is likely that there is some response to its presence. Gene expression patterns can be evaluated using DNA microarray technology to identify the genes involved in solvent tolerance. Gene expression patterns in the presence and absence of organic solvent (hexane or other) can be used to identify solvent responsive genes and their regulatory elements.

As already described herein, promoters are sequence elements in the genome, also known as cis acting sites, that control the expression of downstream coding sequences. They function by recruiting RNA polymerase to the site where it is correctly juxtaposed to initiate transcription. This results in the synthesis of a messenger RNA encoding a protein or a functional RNA molecule that carries out the biochemical function. Promoters have been discovered and characterized that are responsive to small molecule cues, such as metal ions, metabolites, xenobiotics, and etc. The use of an appropriate inducible (or repressible) promoter is an important part of strategies to engineer biosecurity mechanisms in genetically modified algae.

At least two major examples of inducers have been characterized in which a small molecule can induce a promoter to express a gene. In the case of the lac operon, a well studied genetic regulatory system in Escherichia coli, a protein inhibitor of the beta galactosidase gene, termed Lac I, can bind to an operator site upstream of the protein coding sequence. Upon introduction of lactose, the Lac I protein binds lactose molecules, changes confirmation and releases its grip on the operator site of the DNA upstream of the beta galactosidase coding sequence. This in turn allows RNA polymerase to transcribe the beta galactosidase coding sequence and subsequently the enzyme is translated from the resulting messenger RNA molecule. Because lactose is the substrate for beta galactosidase, this system allows the organism to efficiently express this enzyme only when its substrate is present. Another inducible system characterized, this time in eukaryotic cells, is that of steroid hormone receptor activated genes. Steroid receptors of the nuclear receptor family are all transcription factors. Depending upon the intracellular steroid hormone that they bind, they are either located in the cytosol and move to the cell nucleus upon activation, or spend their life in the nucleus waiting for the steroid hormone to enter and activate them. This uptake into the nucleus has to do with Nuclear Localization Signals (NLS) found in a region of the receptor. In most cases this signal is covered up by heat shock proteins (hsp) which bind the receptor until the hormone is present. Upon binding by the hormone the receptor undergoes a conformational change, the hsp come off, and the receptor together with the bound hormone enter the nucleus to act upon transcription.

A promoter responsive to an inducer compound is used to control the expression of an essential gene in some embodiments of the invention. Essential genes are genes whose expression is necessary for the continued growth of the algae. Whether or not a gene is an essential gene depends in part on the nature of the algal species. For example, if the genetically modified alga is substantially incapable of heterotrophic growth, an essential gene can be a gene involved in photosynthesis (e.g., psbA). All of the genes for the photosynthetic complexes have been well documented in the literature and are available as targets for this manipulation (Redding K. et al. J Biol. Chem., 274, p. 10466-73 (1999); Takahashi Y. et al. 1994 Plant Mol. Biol. 24, p. 779-88 (1994)). For example, essential genes from the chloroplast that a photosynthetic alga cannot live without including the Rubisco large and small subunits (rbcL and rbcS) and cytochrome b6/f complex genes petA, petB, and petD, Other potentially useful photosynthesis related genes include RNA polymerase (rpoA, rpoB, and rpoC), ATP synthase genes atpA and atpB, and chlorophyll biogenesis chlB. Interruption of the activity of any of these genes will make the organism less competitive in natural conditions, leading eventually to death of the algae outside of the production facility. Specific genes related to the photosynthetic apparatus can also be useful when using organisms that are capable of mixotrophic or heterotrophic growth since mutant alga can be prepared where the genes have been modified to not contain the genes involved in photosynthesis while maintaining the alga on a fixed carbon source until the gene can be replaced with the same gene under control of the inducible promoter system.

Another example of essential genes are those genes involved in the biosynthesis of various crucial algal metabolites, such as essential amino acids. Examples of essential genes and proteins expressed by essential genes consisting of arg7, meth1, dpd1, ntr3, nadk1, pane, biotin synthetse, dihydrofolate reductase, hemA, hemB, hemL, hemC, hemD, hemE, hemF, hemY, hemG, hemH, hemO, bchG, bchP, and chlG.

In embodiments directed to control of an essential gene, the alga will typically first be modified to remove an essential gene or genes. This can be done using nuclear or chloroplast directed mutagenesis. Alternative approaches can also be utilized where clones are prepared containing the genes under control of the inducible promoter and then removing the endogenous gene by targeted mutagenesis. Alternatively, other lethal metabolic mutations to other essential genes such as those involved in cell wall synthesis or respiration can be made (Cohn M. et al. Mol. Gen. Genet. 249(2):179-84 (1995); Remade C. et al., Proc. Natl. Acad. Sci. USA. 21; 103(12):4771-6 (2006). The key is to make sure these lethal mutants can be “rescued” by the expression cassette including the essential gene under control of a promoter activated by the inducer compound. The term rescued refers to the introduced cassette replacing the gene targeted so the cell can function in the presence of the inducer.

Promoters that activate a gene in the presence of an inducer compound can also be used with other, non-essential genes to provide a biosecure alga. For example, in another embodiment of the invention, the alga can include an antitoxin gene operably linked to a promoter system that is active only in the presence of an inducer compound. In this embodiment, the alga also includes a toxin gene that is constitutively expressed so that the alga will only survive if the inducer is present in the algae culture to stimulate formation of the antitoxin. Promoters can also be used to activate regulatory genes that have an effect on essential genes or other activities that have a positive effect on cell growth. For example, a biosecure alga can be prepared in which the promoter is operatively linked to an anti-apoptotic regulator gene, or the mitochondrial fission inhibitor FIS1.

In another aspect, the present invention provides a biosecure alga in which the genetically modified alga is transformed to include a lethal gene that is operably linked with a promoter system that is inactive only in the presence of a repressor compound. Essentially, this embodiment of the invention involves a method of control that is the reverse of that described previously in which an inducer activates a promoter associated with an essential gene. In this embodiment, the repressor compound functions to inhibit the activity of a promoter that is operably linked to a lethal gene. The repressor compound can be any of the compounds described as being suitable for use as inducer compounds. While the compounds are the same, they can have a different effect depending on the transcription factors and/or promoter chosen to be operably linked to the lethal gene.

A lethal gene, as used herein, refers to a gene whose expression will have a substantially negative effect on the growth and/or survival of the cell. For example, expression of a lethal gene can result in the destruction of the cell (cytotoxicity) or merely the cessation of growth of the cell (cytostasis), depending on the specific lethal gene and the level at which it is expressed. A wide variety of lethal genes are known to those skilled in the art. Examples of lethal genes include genes expressing various cell toxins, such as ccdB, pare, pemK, doc, mazF, relE, and defensins. Since a wide variety of bacterial toxins are known, and the mitochondria and chloroplasts in alga are of bacterial original, the genes for many bacterial toxins provide suitable lethal genes for use in biosecure alga. In cases where the toxin is a toxin effecting the mitochondria or chloroplast of an alga, the N-terminus of the protein expressed by the gene should include a transit peptide to allow the expressed toxin to be targeted to the organelle. A lethal gene can also be a gene that expresses a siRNA specific for an essential gene of the genetically modified alga.

Another example of lethal genes that can be incorporated into biosecure alga include genes whose activation leads to programmed cell death (PCD), also referred to as apoptosis. Examples of genes whose activation results in programmed cell death include pro-apoptotic regulatory genes, p53-induced genes, and PCD-executor genes. Expression of an RNA interference construct (miRNA or siRNA) specific for an anti-apoptotic PCD-regulatory gene can also result in apoptosis. Specific examples of algal pro-apoptotic PCD regulators include algal gene homologs encoding the programmed cell death proteins (PDCD) PDCD2 and PDCD5. Programmed cell death has also been linked to mitochondrial fission caused by DNM1. Accordingly, another example of a lethal gene is DNM1, based on its ability to cause mitochondrial fission.

Further examples of lethal genes that can be incorporated into biosecure algae include genes expressing lytic enzymes or factors. Expression of the lytic enzymes or factors results in cell death as a result of cell lysis. Viral lytic enzymes can be used, and include chlorovirus vAL-1, Paramecium bursaria Chlorella virus (PBCV-1) chitosanase, PBCV-1-chitinase, PBCV-1-β-1,3-glucanase, and viral encoded endolysins. Lysis factors include P22 gp15, and λ Rz. The lysis gene should include a transit peptide such as the periplasmic targeting signal sequence of the Chiamydomonas reinhardtii arylsulfatase or periplasmic carbonic anhydrase proteins to enable secretion of the expressive lytic enzyme or factor mentioned above into the periplasmic space. The lethal genes expressing lytic enzymes or factors can be operatively linked to a repressible promoter so that cell lysis occurs only in the absence of the repressor compound

In another embodiment of the invention, the biosecure alga is modified to include lytic enzymes or factors that lack a transit peptide. These lytic enzymes or factors are constitutively expressed, but are unable to contact the cell wall. The alga of this embodiment also includes a holin gene under the control of a repressible promoter. In the absence of a repressor compound, the holin gene is expressed, resulting in the formation of holes in the cell membrane. These holes allow the lytic enzymes or factors to reach the cell wall, resulting in cell lysis and death. In such a situation, while the lytic enzymes and factors cause the actual cell lysis, the holin gene can be referred to as the lethal gene as its expression triggers actual cell lysis and death.

In practice, the biosecure algae of the present invention are rendered biosecure by modifying the algae to require something that is only provided within an artificial environment. In additional embodiments of the invention, the artificial algal culture provides something other than an inducer or repressor compound to distinguish it from a natural environment. For example, the alga may be genetically modified to lack one or more of the genes involved in repair of damage from ultraviolet light. Genes involved in ultraviolet repair include the photoreactivation and nucleotide excision repair gene systems. Algae modified to have a substantially decreased ability to repair damage from ultraviolet light can then be grown in an artificial algal culture in which they are shielded from full exposure to ultraviolet light. Shielding from ultraviolet light can be provided by covering the pond in which the algae grow with buoyant translucent or transparent spheres or by including a non-toxic UV absorbing compound in the alga culture. Alternately, the algae can be cultured in a tank made from a material that provides full or partial UV protection.

The artificial environment can also be modified to include miRNA or siRNA that interferes with expression of a lethal gene. The miRNA or siRNA are targeted to a lethal gene expressed by algae grown in the artificial algal culture to allow the algae to inhibit the lethal gene so long as the algae remain in the artificial environment. For example, an alga can be modified to overexpress a cytoplasmic lipase or phospholipase, and the biosecure alga is then raised in an artificial algal culture including miRNA or siRNA specific for the cytoplasmic lipase or phospholipase.

The present invention also encompasses various other aspects related to the biosecure alga described herein. For example, the present invention includes methods of growing biosecure algae that have been modified as described herein in an artificial algae culture system, as well as methods of making the biosecure algae according to any one of the methods described herein.

The present invention is illustrated by the following examples. It is to be understood that the particular examples and the materials, amounts, and procedures described therein are merely representative of aspects of the invention, and are to be interpreted broadly in accordance with the scope and spirit of the invention as set forth herein.

### EXAMPLES

### Example 1

### Regulating Gene Expression in Chloroplasts Using Inducible Promoter

The chloroplast genome of Chlamydomonas reinhardtii (model system) is transformed by particle bombardment with a plasmid containing an antibiotic (spectinomycin/streptomycin) resistance selectable marker gene (aadA) and an inducible promoter gene. In addition to the aadA and inducible promoter, the transforming plasmid contains an inducer-driven psbA gene, which is required for assembly and function of the Photosystem II complex that drives water oxidation. The plasmid will be integrated by homologous recombination initially into a psbA deletion mutant, which has to be maintained heterotrophically on acetate for growth. At least 600 bp of flanking DNA, located on both sides of the integrating gene constructs (aadA, inducer driven-psbA), will be identical to the target genome to insure efficient recombination. Transgenic strains are identified by spectinomycin resistance and are confirmed for the presence of the integrating DNA by diagnostic PCR reactions and gene sequencing. For algae other than Chlamydomonas that lack psbA deletion strains but which can be grown heterotrophically, effective psbA deletion strains can be generated by targeted homologous recombination between the wild-type psbA gene plasmids containing interrupted psbA genes. These strains will then serve as hosts for plasmids containing the (aadA, promoter-psbA) constructs. It is noted that while transgenics not expressing the psbA gene could potentially be sustained on certain reduced carbon sources, the presence of these reduced carbon sources in the environment is limited and is unlikely to sustain facultative growth for long time periods should the organisms escape. To assess the likelihood of heterotrophic growth in non-psbA expressing transgenics, the minimal substrate concentrations are determined that support extended viability. The ability of exogenous compounds to induce expression of the psbA gene in chloroplasts is then confirmed by addition of the inducer and photosynthetic growth in the absence of reduced carbon sources. Successful expression of the psbA gene will allow photosynthetic growth in the absence of acetate. Further optimization of photosynthetic growth will be done by performing inducer dose response and time course assays. The inducer proteins are expressed constitutively under the psbA promoter in Chlamydomonas cells, as a model algal system. Two different vectors for homologous recombination-mediated transformation of Chlamydomonas chloroplast genomes were available.

The promoter element will be synthesized commercially and cloned into the chloroplast transformation vector for homologous recombination into the chloroplast genome using 600 bp of flanking homology to the psbA region of a Chlamydomonas psbA deletion mutant. In this construct, the promoter element drives the expression of the intron-less psbA gene. Transgenic cells will be verified by PCR (both the promoter and reporter constructs) and expression of transgenes (for activator constructs) is verified by RT-PCR. A low level of expression of the psbA gene is sufficient to support photosynthetic growth. It is well documented that a photosynthetic rate greater than 10% of wild-type is required for autotrophic survival (Gokhale and Sayre, Photosystem II. Invited chapter in; Chlamydomonas in the Plant Sciences, volume 2, The Chlamydomonas Sourcebook, Elsevier (2009)).

### Example 2

### Preparation of Flavonoid-Inducible Expression Vectors

Four plasmid vectors were successfully produced that include the genetic information necessary to control either a gene required for oxygenic photosynthetic activity or the GUS (beta-glucuronidase) reporter gene. The reporter gene has the advantage of being easily detected through standardized biochemical assays. Two constructs contain the full target constructs and two lack the control gene (nodD1) required for inducing the response act as a negative control in physiological tests. The confirmed constructs were then grown in larger volumes for moderate yield plasmid extraction.

Transformation, selection and screening. The extracted plasmids were then linearized with a restriction enzyme bound to gold beads then transformed into various strains of Chlamydomonas reinhardtii using a microbiolistic approach (i.e., a gene gun). The linearized plasmid vectors contained sequences homologous to the flanking regions around the psbA gene in the chloroplast of C. reinhardtii. The presence of these two flanking region sequences allowed double crossover, homologous recombination resulting in the insertion of the target sequence between the two homologous regions into the chloroplast genome. The correct insertion of the target gene(s) was confirmed through a selection and a screening process. An initial screen was for selection by antibiotic resistance (in this case spectinomycin); aadA gene encodes spectinomycin resistance. Upon successful insertion of this gene, the algae become resistant to spectinomycin. By linearizing the plasmid prior to transformation, the only mechanism by which this gene can be maintained and expressed is if it is inserted into the genome, as it can no longer replicate. This initial screen confirms that the spectinomycin gene has been inserted.

To confirm the insertion of the other target genes, PCR (polymerase chain reaction) was performed using primers specific to the sequences present in the plasmid construct between the two flanking regions and encoding either the psbA gene or the GUS gene. A positive signal (presence of PCR product) from these PCR reactions confirmed that the target sequence was present in the chloroplast. While a negative reaction (no PCR product) may indicate a false positive insertion event where either the spectinomycin gene incorrectly inserted without the accompanying target genes. Primary PCR screens have shown a positive result for the correct insertion of the target genes from each of the four vectors. More detailed analyses of the first 25 isolates for pP0001 have identified 24 isolates which were confirmed positive for growth on spectinomycin and presence of the psbA gene from the vector plasmid.

### Example 3

### Phenotype Characterization Assay to Screen for the Expression of psbA Gene in Transgenic Algae

A quick assay using natural photosynthetic variable fluorescence to screen for the controlled expression of either psbA or the GUS gene in transgenic algae after induction by various flavonoids has been developed. Natural strains of algae are prone to fluorescent emission of light under conditions of excess illumination. The fluorescence is quenched through biological processes to normal levels in a brief period of time and is thus called “variable fluorescence.” In the absence of psbA, there is no variable fluorescence. Upon induction of psbA gene expression by an inducer molecule (flavonoid or other), the biosecure strain displays photosynthetic activity similar to the wild-type. In the absence of the inducer molecule, the culture will cease producing the psbA protein, lose photosynthetic activity, and switch to a pattern of no variable fluorescence similar to that demonstrated by the psbA deletion. This screen is used for detection of psbA activity. The benefit of this screen is its simplicity, in that it requires no additional reagents or cell processing. Simply incubate induced and control cultures in the dark for 10 minutes, then transfer to cuvettes, and measure variable fluorescence using a fluorometer. Screening for GUS activity requires cells to be broken apart mixed with reagents and then processed in a 96 well plate for GUS activity.

### Example 4

### Regulated Expression of a Programmed Cell Death (PCD) Related Gene to Decrease the Survival of a Genetically Modified Alga in the Natural Environment

The nuclear genome of Chlamydomonas reinhardtii can be transformed with a linearized plasmid containing an algal homolog of a PCD-related gene under the control of a repressible promoter. Recent analyses of genomic sequences of green algae have provided evidence for the presence of a large number of PCD-related sequences including PCD-regulator genes, genes encoding nuclear factors specific to PCD, p53-induced genes and PCD-executor genes (Nedelcu, J Mol Evol 68: 256-268; 2009). PCD is a controlled process of cell death, which is hallmarked by the shrinkage of protoplast, fragmentation of DNA (DNA-laddering) and build up of reactive oxygen species.

Considering that PCD is a functional mechanism for cell death native to algae, stimulating the PCD process by (i) the overexpression of an algal pro-apopototic regulatory gene, (ii) the underexpression of an algal anti-apoptotic regulatory gene, (iii) the overexpression of a p53-induced gene, (iv) the overexpression of a PCD-executor gene, or (v) any combination of the aforementioned strategies, should be highly effective in causing cell death. In artificial algae culture (e.g., a pond environment) the expression of the pro-apoptotic PCD-related gene will be repressed, while it will be induced upon the escape of the genetically modified alga to the natural environment causing effective cell death. Similarly, in the artificial algae culture, the expression of the antisense (RNAi) construct of the anti-apoptotic PCD-related gene will be repressed, but will be induced upon the escape of the genetically modified alga to the natural environment causing effective cell death.

The nuclear genome of Chlorella protothecoides is transformed with a linearized plasmid containing an algal homolog of a proapoptotic PCD-regulator gene under the control of a repressible promoter. In artificial algae culture, the expression of the proapoptotic PCD-regulator gene will be repressed, while it will be induced upon the escape of the genetically modified alga to the natural environment causing effective cell death. Examples of algal proapoptotic PCD regulators include, the algal gene homologs encoding the programmed cell death protein (PDCD) 2 (PDCD2), and 5 (PDCD5) (Nedelcu, J Mol Evol 68:256-268; 2009). The overexpression of PDCD5 in rice has been shown to induce programmed cell death (Attia et al., J. Integr. Plant Biol. 47: 1115-22; 2005). Additional proapoptotic PCD regulators include algal gene homologs encoding for Alix/AIP1 (Apoptosis linked gene (ALG)-2-interacting protein X/apoptosis-linked gene 2-interacting protein 1); the gene associated with retinoic-interferon-induced mortality 19 (GRIM-19); genes encoding for proteins containing NACHT domains; or those encoding for the CAS (cellular apoptosis susceptibility) protein and the chromosome-segregation protein (CSE1) (Nedelcu, J Mol Evol 68:256-268; 2009).

The nuclear genome of Chlorella protothecoides will also be transformed with a linearized plasmid containing an RNA interference (RNAi) construct targeted to the algal homolog of an anti-apoptotic PCD-regulator gene under the control of a repressible promoter. In artificial algae culture the expression of the antisense (RNAi) construct of the anti-apoptotic PCD-related gene will be repressed, while it will be induced upon the escape of the genetically modified alga to the natural environment causing effective cell death. Examples of algal anti-apoptotic PCD regulators include the algal gene homologs encoding the defender against death (DAD) proteins, mutations of which can cause cell death (Nakashima et al., Mol Cell Biol 13: 6367-74; 1993). The dad1 Chlamydomonas homolog has been shown to be down-regulated under UV-induced PCD (Moharikar et al., J Biosci 32:261-270; 2007). Additional examples for anti-apoptotic PCD regulators include the algal gene encoding for the apoptosis antagonizing transcription factor (AATF) and for proteins with Mlo domains (Nedelcu, J Mol Evol 68: 256-268; 2009).

The nuclear genome of Chlorella protothecoides will be transformed with a linearized plasmid containing an algal homolog of a p53-induced gene under the control of a repressible promoter to control the propagation of genetically modified alga in the natural environment, in a strategy similar to that described for use of the ccdB gene. Examples of p53-induced genes that have homologs in algae including the p53-induced gene (PIG) 7 and PIG8 (Nedelcu, J Mol Evol 68, 256-268; 2009). The PIG8 (also known as EI24 etoposide-induced 2.4) transcript has been shown to be induced during PCD in green algae (Nedelcu, FEBS Lett 580: 3013-17; 2006).

The nuclear genome of Chlorella protothecoides will be transformed with a linearized plasmid containing an algal homolog of a PCD-executor gene under the control of a repressible promoter in to control the propagation of genetically modified alga in the natural environment. Examples of PCD-executor genes that have homologs in algae include genes encoding for metacaspases types I and II (Nedelcu, J Mol Evol 68, 256-268; 2009). Metacaspases are a class of cysteine proteases that catalyze the cleavage of peptide bonds at basic residues during PCD (Gonzalez et al., Int J Parasitol 37: 161-172; 2007). Additional examples of PCD-executor genes are endonucleases involved in DNA fragmentation that contribute to the DNA laddering hallmark effect of PCD. Algal homologs of proteins with DNA/RNA nonspecific endonuclease domains (EndoG type) have been identified (Nedelcu, J Mol Evol 68, 256-268; 2009). Further examples of PCD-executor genes includes those algal genes encoding for proteins with engulfment and cell motility (ELM) domains.

The mitochondrial genome of Chlorella protothecoides will be transformed with a linearized plasmid containing an algal homolog of a mitochondrial EndoG type endonuclease under the control of a repressible promoter to control the propagation of genetically modified alga in the natural environment, in a strategy similar to that described for the ccdB gene.

### Example 5

### Regulated Expression of a Virus-Encoded Lysis Gene to Decrease the Survival of a Genetically Modified Alga in the Natural Environment

The nuclear genome of Chlorella protothecoides will be transformed with a linearized plasmid containing a gene encoding for a viral lytic enzyme or lysis factor which has been codon optimized for expression in the nucleus of Chlorella protothecoides. The lysis gene will contain a native or engineered transit peptide for secretion to the periplasmic space, and will be expressed under the control of a repressible promoter. Studies have shown the effectiveness of viral-encoded lytic enzymes in breaking down cell wall material or in causing cell lysis of green algae and cyanobacteria. Sugimoto et al., FEBS Lett. 559: 51-56 (2004). An example of a viral-encoded lytic enzyme is chlorovirus vAL-1. The vAL-1 enzyme can degrade C. protothecoides 211-6 cell wall. Sugimoto et al., FEBS Letters. 559: 51-56 (2004). Using TLC, Sugimoto et al. showed degradation products resulted from Chlorella cell wall materials (CWM) treated with vAL-1. CWM of Chlorella strain NC64A, C. protothecoides 211-6, and C. vulgaris C-135 were evaluated.

Other examples of viral-encoded lytic enzymes include Paramecium bursaria Chlorella virus (PBCV-1) chitosanase, PBCV-1-chitinase, PBCV-β-1.3-glucanase and viral-encoded endolysins. Accessory lysis factors include P22, gp15 and λ Rz that have been shown to assist in lysis of the cell wall in bacteria (Berry et al., Mol Microbiol 70: 341-51; (2008); Liu and Curtiss, PNAS, 106(51): 21550-4 (2009). The repressible expression of one or more lytic genes may be executed alone or in combination with the repressible expression of one or more accessory lysis factors. While in artificial algae culture the expression of the lysis gene(s) or factor(s) is repressed, the lysis genes will be expressed upon the escape of the genetically modified alga to the natural environment causing effective cell lysis, thereby preventing the propagation of the genetically modified algal species.

The nuclear genome of Chlorella protothecoides will also be transformed with a linearized plasmid containing a bacteriophage-encoded holin gene codon-optimized for nuclear expression in Chlorella protothecoides, expressed under the control of a repressible promoter. Hans are small membrane spanning proteins that cause nonspecific holes in the cell membrane (Young and Bläsi, FEMS Microbiology Reviews 17: 191-205; 1995). It has been shown that the accumulation of holin is lethal to bacteria and yeast (Garrett et al., J Bact 172: 7275-77 (1990)). This engineered algal strain containing the holin gene, will be transformed with a viral-encoded lysis gene (with or without the co-expression of auxiliary lysis factors) lacking a periplasmic or secretory signal sequence, expressed under a constitutive promoter. Upon escape of the genetically modified algae outside artificial algae culture, the expression of the holin gene will be induced causing a reduction in cell viability and ultimately cell death. Holin expression allows the lytic enzyme (and/or factors) to come in contact with the cell wall via the holes in the cell membrane and elicit the cell lysis process. An example of a lysis gene lacking secretory signals is bacteriophage-encoded endolysin. An advantage to combining use of a holin gene under control of a repressible promoter together with the constitutive expression of lysis genes lacking a periplastmic or secretory signal sequence is that the lysis proteins will accumulate to high levels, and their sudden release by holin expression will result in very rapid lethality for the cell.

### Example 6

### UV Repair Mutations to Provide Biocontainment

A system which grows genetically modified algae deficient in or lacking in the functionality of one or more of the genes involved in ultraviolet repair within open top bioreactors manipulated to minimize ultraviolet (UV) penetration using UV absorbing chemical additives in the media, and or through the use of white light transparent, but UV blocking materials at the water surface. In this system, passive escape or active removal of UV intolerant algae to other compatible water bodies, though ones not actively maintained to reduce ultraviolet light, would be severely impaired by exposure to unfiltered sunlight, rendering them non-competitive and non-viable.

Target UV repair gene systems include photoreactivation and or nucleotide excision repair. Photoreactivation involves the enzyme photolyase (EC 4.1.99.3), which associates and directly reverses cyclobutane pyrimidine dimers (CPD) or 6,4 pyrimidine-pyrimidone lesions formed in DNA exposed to high energy ultraviolet light radiation (UVA+UVB). Nucleotide excision repair excises a region surrounding a CPD and directs DNA synthesis of the molecule. Dimer formations are additive to UV exposure and are distributed throughout all genomes (nuclear, mitochondrial and chloroplastic). Lesions alter the structure of DNA and, if unrepaired, inhibit polymerases, disrupting metabolic functioning, and arrest cell replication. Loss of UV repair mechanisms would severely impair the viability of a manipulated algal production strains outside of UV manipulated production ponds, assuring containment of the genetically modified algae. Methods to disrupt gene function could include classical mutagenic approaches (UV or chemical mutagen) paired strain screening of UV intolerant phenotypes, site-directed mutagenesis using either an insertion of a nonsense or missense mutation to target gene, or a RNAi strategy paired to biolistics or similar transformation technique to reduce transcript levels within the transformed algal cell.

Low cost chemicals that are benign to algae could include many types of water soluble phenolic acids (e.g. humic or tannic acid, flavonoids) or other phenylpropanoid type compounds that at low concentrations in pond media, would absorb and filter out radiation in the ultraviolet spectra. Similarly, floating translucent or transparent hollow plastic (e.g., polycarbonate, mylar, polypropylene, low density polyethylene) or glass spheres, 0.1 to 10 cm in diameter, that are composed, coated or blended with materials that strongly absorb ultraviolet light, could be deployed at sufficient densities at the surface of ponds to shield a UV vulnerable pond algae from ultraviolet light without a significant reduction in photosynthetically active radiation. UV vulnerable algae would be kept healthy and productive by way of excluding exposures to harmful ultraviolet light.

### Example 7

### Cytoplamic Lipase or Phospholipase for Biocontainment

A system that grows algae, genetically manipulated to overexpress a cytoplasmic lipase or a phospholipase, can be used to provide biosecure algae. Use of RNAi technology could be used as a means to control the lipase by adding microRNA (miRNA) and small interfering RNAs (siRNA) directly to ponds. These small RNA molecules would be taken up by pond algae, eliminating the expression of the lipase. Upon escape from production ponds, overexpression of the lipase in the recombinant algae would attack the integrity of lipid-based cell membranes of the cell wall and organelles, destabilizing metabolism, osmotic stability and cell viability. Additional support for this system can be found in the following references:
(1) Sancar A. (2003). “Structure and function of DNA photolyase and cryptochrome blue-light photoreceptors”. Chem Rev 103 (6): 2203-37
(2) Hutschison, C. A., Philipps, S., Edgell, M. H., Gillham, S., Jahnke, P., Smith, M. (1978) Mutagenesis at a Specific Position in a DNA Sequence. J. Biol. Chem. 253: (18) 6551-6560
(3) Fire A, Xu S, Montgomery M, Kostas S, Driver S, Mello C (1998). “Potent and specific genetic interference by double-stranded RNA in Caenorhabditis elegans”. Nature 391 (6669): 806-11.

### Example 8

### Regulated Expression of a Lethal Gene to Decrease Survival of Genetically, Modified Alga in a Natural Environment

The nuclear or mitochondrial genome of Chlorella protothecoides will be transformed with a plasmid containing the ccdB gene from the F plasmid of E. coli under the control of a repressible promoter. The product of the ccdB gene is toxic to E. coli as it affects the functioning of E. coli DNA gyrase (Bernard and Couturier, 1992). Since the eukaryotic organelles mitochondria and chloroplast are of bacterial origin, the CcdB toxin is expected to affect activity of mitochondrial and chloroplastic DNA gyrase. The N-terminus of CcdB protein will have the required transit peptide to target it to Chlorella protothecoides mitochondrion. In transgenic algae, expression of CcdB will be repressed under production conditions using a chemically repressible promoter. If the alga is removed or escapes from the production system, expression of ccdB will be activated owing to the absence of the repressor resulting in production of CcdB toxin. The CcdB toxin will poison the mitochondrial DNA gyrase resulting in algal death. This system will ensure that the genetically modified algae cannot escape and damage the environment.

The nuclear or mitochondrial genome of Chlorella protothecoides will be transformed with a plasmid containing the ccdB/ccdA toxin/antitoxin (TA) genes from the F plasmid of E. coli. The ccdB gene codes for a toxin that affects activity of E. coli DNA gyrase and ccdA encodes an antitoxin that binds to CcdB and keeps it inactive (Miki et al., 1984; Bernard and Couturier, 1991 & 1992). While the expression of ccdB will be under the control of a constitutive promoter, expression of ccdA will be controlled by a chemically inducible promoter. The N-terminus of CcdA and CcdB proteins will have the required transit peptides to target these proteins to the Chlorella protothecoides mitochondrion. The transgenic algae will express the toxin (CcdB) and antitoxin (CcdA) under production conditions due to the presence of the induced chemical (i.e., CcdA). If the alga is removed or escapes from the production system, expression of ccdA will be stopped due to absence of the inducer, whereas the expression of ccdB will not be affected. As a result the CcdB toxin will accumulate in mitochondria and poison the mitochondrial DNA gyrase resulting in algal death. This system will ensure that the genetically modified organisms cannot escape and damage the environment.

The parE gene from E. coli plasmid RK2/RP4 codes for a toxin that affects E. coli DNA gyrase activity (Jiang et al., 2002) can also be used to control growth and spread of transgenic alga in natural environments following the strategies described for use of the ccdB gene.

The parE/parD toxin/antitoxin system from E. coli plasmid RK2/RP4 (Roberts and Helinski, 1992) can also be used to control growth and spread of transgenic alga in natural environments following the strategies described for use of the ccdB gene and ccdA antitoxin.

The pemK gene from E. coli plasmid R100 codes for a toxin that inhibits protein synthesis by cleaving mRNAs at specific sites (Zhang et al., 2004) can also be used to control growth and spread of transgenic alga in natural environments following the strategies described for use of the ccdB gene.

The pemK/pemI toxin/antitoxin system from E. coli plasmid R100 (Tsuchimoto et al., 1988; Zhang et al., 2004) can also be used to control growth and spread of transgenic alga in natural environments following the strategies described for use of the ccdB gene and ccdA antitoxin.

The doc gene of E. coli plasmid prophage P1 codes for a protein that inhibits translation elongation by associating with the 30s ribosomal subunit (Liu et al., 2008). This gene can also be used to control growth and spread of transgenic alga in natural environments following the strategies described for use of the ccdB gene.

The phd-doc, antitoxin-toxin system of E. coli plasmid prophage (Lehnherr et al., 1993) can also be employed to control growth and spread of transgenic alga in natural environments following the strategies described for use of the ccdB gene and ccdA antitoxin gene.

The E. coli gene mazF codes for a toxin that inhibits protein synthesis by cleaving mRNAs at specific sites (Christensen et al., 2003) can also be used to control growth and spread of transgenic alga in natural environments following the strategies described for use of the ccdB gene.

The E. coli mazF/mazE toxin/antitoxin system (Metzger et al., 1988; Aizenman et al., 1996) can also be used to control growth and spread of transgenic alga in natural environments following the strategies described for use of the ccdB gene and ccdA antitoxin gene.

The E. coli gene relE codes for a toxin that inhibits protein synthesis by cleaving mRNAs (Christensen et al., 2001) can also be recruited to control growth and spread of transgenic alga in natural environments following the strategies described for use of the ccdB gene.

The E. coli relE/relB toxin/antitoxin system (Gotfredsen and Gerdes, 1998) can also be used to control growth and spread of transgenic alga in natural environments following the strategies described for use of the ccdB gene and ccdA antitoxin.

In addition other bacterial toxins and toxin/antitoxin systems can be used to control growth and spread of transgenic alga in natural environments following the strategies described for use of the ccdB gene and the combined use of the ccdB gene and ccdA antitoxin.

The genes coding for antimicrobial peptides, commonly called defensins can also be used to control growth and spread of transgenic alga in natural environments. Defensins are produced by plants, animals and insects to protect against bacterial, fungal and viral infections. Most of the antimicrobial peptides act by disrupting the integrity of bacterial cell membranes (Yang et al., 2004). Being of bacterial origin, the composition of mitochondrial and chloroplast membranes is similar to bacterial membranes, thus making them susceptible to antimicrobial peptides. The antimicrobial peptides can be employed to control spread and growth of transgenic algae in natural environments following the strategies described for use of the ccdB gene.

The cytoplasmic male sterility (CMS) trait is widely used for hybrid seed production in many crop plants. The CMS trait has been linked to expression of small lethal recombinant peptides (SLRP) in mitochondria. SLRPs are encoded by recombinant open reading frames (ORFs) that arise as a consequence of repeated recombination of mitochondrial genome. SLRPs disrupt mitochondrial function by creating pores in mitochondrial membranes (Rhoads et al., 1995) and cause pollen abortion (i.e. male sterility). Over expression of these peptides in E. coli, yeast and insect cells has been found to be toxic to host cells (Dewey et al., 1988; Huang et al., 1990; Korth et al., 1991; Korth and Levings, 1993). The SLRP encoding ORFs can be used to control spread and growth of transgenic algae in natural environment following the strategies described for use of the ccdB gene. The term “SLRP encoding ORFs” mentioned above includes but is not restricted to recombinant ORFs from bean (orf239) (Mackenzie and Chase, 1990), petunia (pcf) (Hanson et al., 1999), sunflower (orf522) (Horn et al., 1991), sorghum (orf107) (Tang et al., 1996), radish (orf125) (Iwabuchi et al., 1999), brassica (orf224, orf222) (L'Homme et al., 1997) and rice (orf79) (Akagi et al., 1994). In addition, new SLRP encoding ORFs can be created by fusing coding sequences of two or more genes.

### REFERENCES

Aizenman E, Engelberg-Kulka H, Glaser G (1996) An Escherichia coli chromosomal “addiction module” regulated by guanosine [corrected] 3′,5′-bispyrophosphate: a model for programmed bacterial cell death. Proc Natl Acad Sci USA 93: 6059-6063
Akagi H, Sakamoto M, Shinjyo C, Shimada H, Fujimura T (1994) A unique sequence located downstream from the rice mitochondrial atp6 may cause male sterility. Curr Genet. 25: 52-58
Bernard P, Couturier M (1991) The 41 carboxy-terminal residues of the miniF plasmid CcdA protein are sufficient to antagonize the killer activity of the CcdB protein. Mol Gen Genet. 226: 297-304
Bernard P, Couturier M (1992) Cell killing by the F plasmid CcdB protein involves poisoning of DNA-topoisomerase II complexes. J Mol Biol 226: 735-745
Christensen S K, Mikkelsen M, Pedersen K, Gerdes K (2001) RelE, a global inhibitor of translation, is activated during nutritional stress. Proc Natl Acad Sci USA 98: 14328-14333
Christensen S K, Pedersen K, Hansen F G, Gerdes K (2003) Toxin-antitoxin loci as stress-response-elements: ChpAK/MazF and ChpBK cleave translated RNAs and are counteracted by tmRNA. J Mol Biol 332: 809-819
Dewey R E, Siedow J N, Timothy D H, Levings C S, 3rd (1988) A 13-kilodalton maize mitochondrial protein in E. coli confers sensitivity to Bipolaris maydis toxin. Science 239: 293-295
Gotfredsen M, Gerdes K (1998) The Escherichia coli relBE genes belong to a new toxin-antitoxin gene family. Mol Microbiol 29: 1065-1076
Hanson M R, Wilson R K, Bentolila S, Kohler R H, Chen H C (1999) Mitochondrial gene organization and expression in petunia male fertile and sterile plants. J Hered 90: 362-368
Horn R, Kohler R H, Zetsche K (1991) A mitochondrial 16 kDa protein is associated with cytoplasmic male sterility in sunflower. Plant Mol Biol 17: 29-36
Huang J, Lee S H, Lin C, Medici R, Hack E, Myers A M (1990) Expression in yeast of the T-urf13 protein from Texas male-sterile maize mitochondria confers sensitivity to methomyl and to Texas-cytoplasm-specific fungal toxins. Embo J 9: 339-347
Iwabuchi M, Koizuka N, Fujimoto H, Sakai T, Imamura J (1999) Identification and expression of the kosena radish (Raphanus sativus cv. Kosena) homologue of the ogura radish CMS-associated gene, orf138. Plant Mol Biol 39: 183-188
Jiang Y, Pogliano J, Helinski D R, Konieczny I (2002) ParE toxin encoded by the broad-host-range plasmid RK2 is an inhibitor of Escherichia coli gyrase. Mol Microbiol 44: 971-979
Korth K L, Kaspi C I, Siedow J N, Levings C S, 3rd (1991) URF13, a maize mitochondrial pore-forming protein, is oligomeric and has a mixed orientation in Escherichia coli plasma membranes. Proc Natl Acad Sci USA 88: 10865-10869
Korth K L, Levings C S, 3rd (1993) Baculovirus expression of the maize mitochondrial protein URF13 confers insecticidal activity in cell cultures and larvae. Proc Natl Acad Sci USA 90: 3388-3392
L'Homme Y, Stahl R J, Li X Q, Hameed A, Brown G G (1997) Brassica nap cytoplasmic male sterility is associated with expression of a mtDNA region containing a chimeric gene similar to the pol CMS-associated orf224 gene. Curr Genet. 31: 325-335
Lehnherr H, Maguin E, Jafri S, Yarmolinsky M B (1993) Plasmid addiction genes of bacteriophage P1: doc, which causes cell death on curing of prophage, and phd, which prevents host death when prophage is retained. J Mol Biol 233: 414-428
Liu M, Zhang Y, Inouye M, Woychik N A (2008) Bacterial addiction module toxin Doc inhibits translation elongation through its association with the 30S ribosomal subunit. Proc Natl Acad Sci USA 105: 5885-5890
Mackenzie S A, Chase C D (1990) Fertility Restoration Is Associated with Loss of a Portion of the Mitochondrial Genome in Cytoplasmic Male-Sterile Common Bean. Plant Cell 2: 905-912
Metzger S, Dror I B, Aizenman E, Schreiber G, Toone M, Friesen J D, Cashel M, Glaser G (1988) The nucleotide sequence and characterization of the relA gene of Escherichia coli. J Biol Chem 263: 15699-15704
Miki T, Chang Z T, Horiuchi T (1984) Control of cell division by sex factor F in Escherichia coli. II. Identification of genes for inhibitor protein and trigger protein on the 42.84-43.6 F segment. J Mol Biol 174: 627-646
Rhoads D M, Levings C S, 3rd, Siedow J N (1995) URF13, a ligand-gated, pore-forming receptor for T-toxin in the inner membrane of cms-T mitochondria. J Bioenerg Biomembr 27: 437-445
Roberts R C, Helinski D R (1992) Definition of a minimal plasmid stabilization system from the broad-host-range plasmid RK2. J Bacteriol 174: 8119-8132
Tang H V, Pring D R, Shaw L C, Salazar R A, Muza F R, Yan B, Schertz K F (1996) Transcript processing internal to a mitochondrial open reading frame is correlated with fertility restoration in male-sterile sorghum. Plant J 10: 123-133
Tsuchimoto S, Ohtsubo H, Ohtsubo E (1988) Two genes, pemK and pemI, responsible for stable maintenance of resistance plasmid R100. J Bacteriol 170: 1461-1466
Yang D, Biragyn A, Hoover D M, Lubkowski J, Oppenheim J J (2004) Multiple roles of antimicrobial defensins, cathelicidins, and eosinophil-derived neurotoxin in host defense. Annu Rev Immunol 22: 181-215
Zhang J, Zhang Y, Zhu L, Suzuki M, Inouye M (2004) Interference of mRNA function by sequence-specific endoribonuclease PemK. J Biol Chem 279: 20678-20684

### Example 9

### Expression of Mutant Forms of Endogenous Algal Genes to Decrease the Survival of Genetically Modified Alga in a Natural Environment

Multi-subunit proteins, such as ATP synthase, RNA polymerase, DNA polymerase and etc, that are required for survival of algae can be targeted by molecular approaches to reduce the fitness and survival of transgenic algae in natural environments. Mutation of one or more of the subunits of these genes can be carried out to substantially reduce or eliminate the activity of the expressed multi subunit protein. An advantage of modifying a multi subunit protein is that the function of a multi subunit protein can be easily disrupted by overexpressing one or more mutant subunits. For example the mammalian mitochondrial atp synthase consists of 16 different subunits. The alpha (α) and beta (β) subunits are each present in three copies. Hence over expression of a mutant copy of alpha subunit can reduce the probability of assembling a fully functional atp synthase protein by 87.5% (the probability of assembling a fully functional protein will be ½*½*½). In addition if a mutant form of beta sub unit is also over expressed, it will further reduce the probability of assembling a fully functional protein by 87.5%. Thus expression of a mutant sub unit will behave as a dominant mutation.

Expression of mutated forms of the multi subunit proteins can result in a substantial decrease in activity as a result of inactive forms of the protein competing with active forms. This provides a means for targeting the activity of essential genes in a biosecure alga. For example, a repressible promoter operatively linked to a mutated gene for a multi subunit protein can be included in an alga to result in a substantial decrease in the activity of the protein in the absence of the repressor compound. Accordingly, regulated over-expression of mutant subunits that make the fully assembled protein non functional can be used to prepare biosecure alga, thereby preventing the spread of transgenic algae into the wild.

### Example 10

### Regulated Expression of Genes that Regulate Programmed Cell Death Through Mitochondrial Fission

Programmed cell death in unicellular organisms like yeast has been linked to mitochondrial fission. Yeast proteins DNM1 and FIS1 that regulate mitochondrial fission are highly conserved and their homologs have been found in plants, humans and algae. While DNM1 promotes mitochondrial fission, FIS1 inhibits mitochondrial fission (Mozdy et al., J Cell Biol 151: 367-380 (2000), Fannjiang et al., Genes and Devt 18: 2785-2797 (2004)). Hence over expression of DNM1 or suppression of FIS1 can be employed to control spread of transgenic algae in wild. The nuclear genome of Chlorella protothecoides will be transformed with a plasmid containing the algal homolog of the yeast dnm1gene under the control of a repressible promoter. In transgenic algae, expression of dnm1 will be repressed under production conditions using a chemically repressible promoter. If the alga is removed or escapes from the production system, expression of dnm1 will be activated owing to absence of the repressor resulting in production of DNM1 protein. The DNM1 protein will promote mitochondrial fission causing algal mortality. This system will ensure that the genetically modified organisms cannot escape and damage the environment. Such a system would work especially well for cyanobacterial production strains.

The nuclear genome of Chlorella protothecoides will be transformed with a plasmid containing the antisense transcript of algal homolog of yeast fis1 gene under the control of a repressible promoter. In transgenic algae, expression of fis1 antisense transcript will be repressed under production conditions using a chemically repressible promoter. If the alga is removed or escapes from the production system, expression of Fis1 antisense transcript will be activated owing to absence of the repressor resulting in repression of Fis1 expression. The inhibition of expression of algal homolog of fis1 will promote mitochondrial fission causing algal mortality. This system will ensure that the genetically modified organisms cannot escape and damage the environment.

### Example 11

### Example for Barstar/Barnase BioSecurity System in Chlorella Protothecoides

Barstar and Barnase genes can be used for engineering Chlorella protothecoides to include a biosecurity system. This strategy can be applied using either nuclear transformation or chloroplast transformation of Chlorella protothecoides. A nuclear transformation vector is constructed and used to first create an algal strain that can inducibly express an rbcS transit peptide:: T7 RNA polymerase gene from a chemical inducible promoter. That strain is subsequently transformed with a chloroplast transformation vector that is constructed with two cassettes, one for Barnase and one for Barstar. The Barnase gene used encodes a 110 amino acid RNase that is cytotoxic when present in a biological system in the absence of Barstar protein. The Barstar gene used encodes a 90 amino acid protein that is a specific inhibitor of Barnase. Synthetic coding region genes are synthesized for both Barstar and Barnase and used as templates for making chloroplast transformation vectors.

### Barnase Synthetic Gene:

The Barnase synthetic gene is synthesized in the context of a chloroplast gene expression cassette (5′ untranslated leader or promoter::Bamase protein coding sequence::3′ untranslated leader). The Barnase protein coding sequence is also designed using algal chloroplast codon preferences. The nucleotide sequence is actually made from two synthetic genes that overlap at a BstEII restriction endonuclease cleavage site. This allows the manufacturer to synthesize and propagate each of the two portions of the Barnase gene cassette without the possibility of encoding a functional Barnase protein, which may kill bacterial host cells used in the cloning and plasmid amplification process. The 5′ untranslated region (UTR) and 3′ UTR of the Chlorella protothecoides chloroplastic atpF gene is used to constitutively express the Barnase synthetic gene and appropriately terminate transcription in algal chloroplasts.

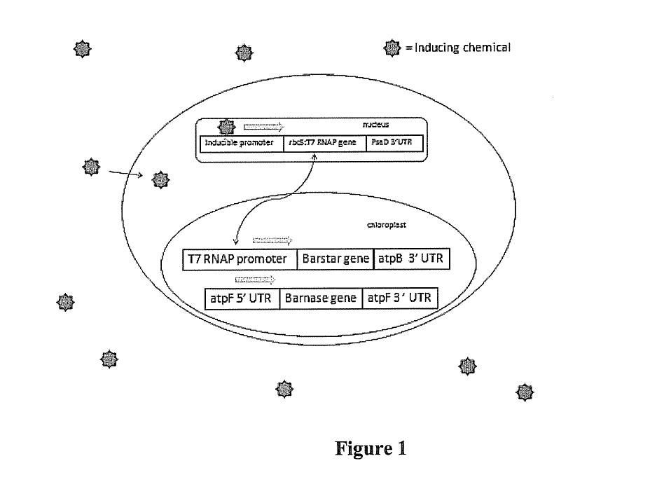

The Barstar synthetic gene is designed using Chlorella protothecoides chloroplast codon preferences. The chloroplast gene cassette for Barstar will use an inducible promoter system to drive inducible expression of the Barstar protein. The promoter juxtaposed upstream of the Barstar protein coding sequence will be a bacteriophage T7 promoter. The 3′UTR juxtaposed downstream of the Barstar protein coding sequence will be the 3′UTR from the Chlorella protothecoides chloroplast atpB gene for transcription termination. See FIG. 1. The algal strain to be used for transformation of the inducible Barstar gene cassette will be a strain that is already modified with a nuclear transformation vector containing a gene cassette that uses a chemical inducible promoter to drive expression of an engineered bacteriophage T7 RNA polymerase competent for post-translation import into algal chloroplasts in a manner that retains its functional ability to transcribe genes downstream of a T7 RNA polymerase promoter. This T7 RNA polymerase gene is engineered using Chlorella protothecoides nuclear codon preferences to encode the polymerase as well as the transit peptide of the Chlorella protothecoides rbcS (small subunit of ribulose bisphosphate carboxylase/oxygenase) gene homolog. The nuclear cassette also uses the transcriptional terminator of the Chlorella protothecoides nuclear psaD gene.

An algal strain that harbors two chloroplast transgene cassettes and one nuclear transgene cassette will be viable as long as the chemical that induces expression of the T7 RNA polymerase transgene is present and allows steady expression of Barstar protein. This steady stream of available Barstar protein is necessary to inhibit activity of the constitutively expressed Barnase in the chloroplast and preventing it from killing the cell. Genetic elements from the Chlorella protothecoides genome, such as the UTRs for expression of foreign genes in the chloroplast, may be used, as they become identified and characterized from the genomic sequence of Chlorella protothecoides, in lieu of those previously identified and characterized from well studied algal species such as Chlamydomonas species, etc.

References for Barstar and Barnase strategies for engineering biocontainment in oleaginous algae:
Robert W. Hartley (1988) Barnase and Barstar: Expression of its Cloned Inhibitor Permits Expression of a Cloned Ribonuclease. J. Mol. Biol. 202:913-915.
Harry Daniell (2002) Molecular Strategies for Gene Containment in Transgenic Crops. Nature Biotechnology 20:581-586.
Ray, K., Bisht, N. C., Deepak Pental, D., and P. K. Burma (2007) Development of barnase/barstar transgenics for hybrid seed production in Indian oilseed mustard (Brassica juncea L. Czem & Coss) using a mutant acetolactate synthase gene conferring resistance to imidazolinone-based herbicide ‘Pursuit’. Current Science 93(10):1390-1396.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art, including variations using redundant, multiple molecular strategies in the same modified organism, will be included within the invention defined by the claims.

1. A biosecure alga consisting of a genetically modified alga comprising an essential gene that is operably linked to a promoter system that is active only in the presence of an inducer compound.

2. The biosecure alga of claim 1, wherein the alga is a species selected from the group consisting of Chlamydomonas sp., Chlorella sp., Nannochloropsis sp., Synechocystis sp., Synechococcus, Anabaena sp., Cyclotella, Phaeodactylum sp., Crypthicodineum sp., Schizochytridum sp., Haematococcus sp., Arthrospira (Spirulina) sp. and Dunaliella sp.

3. The biosecure alga of claim 1, wherein the alga has been genetically modified to provide increased lipid production.

4. The biosecure alga of claim 1, wherein the alga is substantially incapable of heterotrophic growth and the essential gene is a gene involved in photosynthesis.

5. The biosecure alga of claim 4, wherein the gene involved in photosynthesis is selected from the group of genes consisting of rbcL, rbcS, petA, petB, petD, rpoA, rpoB, rpoC, actP, atpB, psbA, and chlB.

6. The biosecure alga of claim 1, wherein the essential gene is a gene involved in essential amino acid biosynthesis.

7. The biosecure alga of claim 1, wherein the essential gene is selected from the group of genes and proteins expressed by genes consisting of arg7, meth1, dpd1, ntr3, nadk1, pane, biotin synthetase, dihydrofolate reductase, hemA, hemB, hemL, hemC, hemD, hemE, hemF, hemY, hemG, hemH, hemO, bchG, bchP, and chlG.

8. The biosecure alga of claim 1, wherein the essential gene is an anti-apoptotic regulator gene.

9. A biosecure alga consisting of a genetically modified alga comprising a lethal gene that is operably linked with a promoter system that is inactive only in the presence of a repressor compound.

10. The biosecure alga of claim 9, wherein the alga is a species selected from the group consisting of Chlamydomonas sp., Chlorella sp., Nannochloropsis sp., Synechocystis sp., Synechococcus, Anabaena sp., Cyclotella, Phaeodactylum sp., Crypthicodineum sp., Schizochytridum sp., Haematococcus sp., Arthrospira (Spirulina) sp. and Dunaliella sp.

11. The biosecure alga of claim 9, wherein the alga has been genetically modified to provide increased lipid production.

12. The biosecure alga of claim 9, wherein the lethal gene expresses a siRNA specific for an essential gene of the alga.

13. The biosecure alga of claim 12, wherein the essential gene is selected from the group of genes and proteins expressed by genes consisting of arg7, meth1, dpd1, ntr3, nadk1, pane, biotin synthetase, dihydrofolate reductase, hemA, hemB, hemL, hemC, hemD, hemE, hemF, hemY, hemG, hemH, hemO, bchG, bchP, and chlG.

14. The biosecure alga of claim 9, wherein the lethal gene is a pro-apoptotic regulatory gene, a PCD-executor gene, a p53-induced gene, or an RNA interference construction specific for an anti-apoptotic PCD-regulator gene.

15. The biosecure alga of claim 9, wherein the lethal gene is a gene expressing a lytic enzyme or a lytic factor.

16. The biosecure alga of claim 9, wherein the lethal gene is an E. coli gene expressing a toxin and is selected from the group consisting of ccdB, pare, pemK, doc, mazF, relE, and defensins.

17. The biosecure alga of claim 9, wherein the lethal gene is a holin gene, and the nuclear genome of the alga has also been modified to constitutively express one or more lytic genes that do not include a secretory signal sequence.

18. A biosecure alga consisting of a genetically modified alga comprising a constitutively expressed toxin gene and a corresponding antitoxin gene operably linked to a promoter system that is active only in the presence of an inducer compound.

19. The biosecure alga of claim 18, wherein the toxin and antitoxin genes are selected from the group of E. coli toxin/antitoxin pairs consisting of ccdB/ccdA, parE/parD, pemK/pemI, doc/phd, mazF/mazE, and relE/relB or from the toxin/antitoxin pair consisting of Barnase/Barstar from Bacillus species

20. The biosecure alga of claim 18, wherein the toxin and antitoxin genes are the barnase/barstar toxin/antitoxin pair.

21. A method of making a biosecure alga according to claim 1, comprising transforming a genetically modified alga to include an essential gene that is operably linked to a promoter system that is active only in the presence of an inducer compound.

22. A method of making a biosecure alga according to claim 9, comprising transforming a genetically modified alga to include a lethal gene that is operably linked with a promoter system that is inactive only in the presence of a repressor compound.

23. A method of culturing of a genetically modified alga in a biosecure manner, comprising culturing a genetically modified alga including a disrupted UV repair gene in an artificial environment providing low UV exposure.

24. The method of claim 23, wherein the IN repair gene is a gene expressing photolyase or a nucleotide excision repair protein.

25. The method of claim 23, wherein UV exposure is decreased by culturing the alga in a pond covered with buoyant translucent or transparent spheres.

26. The method of claim 23, wherein UV exposure is decreased by including a non-toxic UV absorbing compound in the alga culture.
